(19) United States
(12) Patent Application Publication
(10) Pub. No.: US 2011/0264116 A1
(43) Pub. Date: Oct. 27, 2011

(54) Compressive Denervation Apparatus for Innervated Renal Vasculature

(76) Inventors: Gordon Kocur, Lino Lakes, MN (US); Dave Sogard, Edina, MN (US); Anthony Vrba, Maple Grove, MN (US); Roger Hastings, Maple Grove, MN (US)

(21) Appl. No.: 12/980,948

(22) Filed: Dec. 29, 2010

Related U.S. Application Data
(60) Provisional application No. 61/291,471, filed on Dec. 31, 2009.

(51) Int. Cl. A61B 17/10; A61F 2/82; A61M 29/00

(52) U.S. Cl. 606/139; 606/194; 623/1.15

(57) ABSTRACT

Devices, systems, and methods facilitate modification of renal sympathetic nerve activity using a force generating arrangement. A device for mechanically modifying renal sympathetic nerve activity includes a contact arrangement having a shape that generally conforms to a portion of a renal artery wall and is configured for placement at the renal artery wall portion. A compression arrangement is configured to cooperate with the contact arrangement to place the wall portion of the renal artery in compression sufficient to achieve a desired reduction in renal sympathetic nerve activity. The compression arrangement and the contact arrangement are preferably configured to cooperatively place the wall portion of the renal artery in compression sufficient to irreversibly terminate renal sympathetic nerve activity.

### RELATED PATENT DOCUMENTS

This application claims the benefit of Provisional Patent Application Ser. No. 61/291,471, filed on Dec. 31, 2009, to which priority is claimed under 35 U.S.C. §119(e), and which is incorporated herein by reference.

### TECHNICAL FIELD

The present invention is related to systems and methods for improving cardiac and/or renal function through neuromodulation, including disruption and termination of renal sympathetic nerve activity.

### BACKGROUND

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

### SUMMARY

Devices, systems, and methods of the present invention are directed to modifying renal sympathetic nerve activity using a force generating arrangement. According to embodiments of the present invention, a device for mechanically modifying renal sympathetic nerve activity includes a contact arrangement having a shape that generally conforms to a portion of a renal artery wall and is configured for placement at the renal artery wall portion. The device includes a compression arrangement configured to cooperate with the contact arrangement to place the wall portion of the renal artery in compression sufficient to achieve a desired reduction in renal sympathetic nerve activity. The compression arrangement and the contact arrangement are preferably configured to cooperatively place the wall portion of the renal artery in compression sufficient to irreversibly terminate renal sympathetic nerve activity. In some embodiments, all or at least a portion of the contact arrangement and the compression arrangement is constructed from one or more biodegradable materials.

Embodiments of the present invention are directed to a fastener for mechanically modifying renal sympathetic nerve activity. A fastener of the present invention may include a contact arrangement comprising a first element configured to contact an outer wall of a target vessel and a second element configured to contact an inner wall of the target vessel. At least one of the first and second elements has a collapsible configuration that facilitates passage through an access hole developed in the target vessel wall when in the collapsed configuration. A force generating arrangement is coupled to the contact arrangement and configured to mechanically cooperate with one or both of the first and second elements to place a wall portion of the target vessel in compression sufficient to achieve a desired reduction in renal sympathetic nerve activity. The target vessel is preferably one of the renal artery and the abdominal aorta. The fastener may be configured as, or comprise, a rivet, such as a blind rivet. In some embodiments, all or at least one or more portions of the fastener is constructed from one or more biodegradable materials.

In accordance with other embodiments, a cuff device is configured for placement on the renal artery to mechanically modify renal sympathetic nerve activity. The cuff member is dimensioned to be disposed over an exterior wall portion of a renal artery. The cuff member includes a contact surface configured to engage the exterior wall portion of the renal artery. A compression element is coupled or integral to the cuff member. The compression element and cuff member cooperate to place the wall portion of the renal artery in compression sufficient to achieve a desired reduction in renal sympathetic nerve activity. In some embodiments, all or at least one or more portions of the cuff device is constructed from one or more biodegradable materials.

In further embodiments, an apparatus for mechanically modifying renal sympathetic nerve activity includes a stent configured for endoluminal deployment within the renal artery and a filament configured for placement around an exterior wall portion of the renal artery and at a location proximate the stent. Cooperation between the stent and contraction or shortening of the filament places the wall portion of the renal artery in compression sufficient to achieve a desired reduction in renal sympathetic nerve activity. In some embodiments, all or at least one or more portions of the stent and/or filament is constructed from one or more biodegradable materials.

According to some embodiments, a device for mechanically modifying renal sympathetic nerve activity includes a contact arrangement having a shape that generally conforms to a portion of a renal artery wall and is configured for placement at the renal artery wall portion. The device includes a compression arrangement configured to cooperate with the contact arrangement to place the wall portion of the renal artery in compression sufficient to achieve a desired reduction in renal sympathetic nerve activity. In some embodiments, all or at least one or more portions of the device is constructed from one or more biodegradable materials.

The device further includes a treatment arrangement coupled to the contact arrangement. The treatment arrangement is configured to deliver a treatment agent to the renal artery wall portion to facilitate reduction in renal sympathetic nerve activity. For example, the treatment arrangement may include an electrode arrangement configured to receive energy from a source remote from the renal artery wall portion and generate heat that is communicated to the renal artery wall portion. The treatment arrangement may include a mechanism for delivering a pharmacological agent to the renal artery wall portion, such as a neurotoxin or venom.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3 illustrates various tissue layers of the wall of the renal artery;

FIG. 4A illustrates a compression arrangement deployed at the wall of the renal artery shown in FIG. 3 and in a pre-compressed configuration in accordance with embodiments of the present invention;

FIG. 4B illustrates a compression arrangement deployed at the wall of the renal artery shown in FIG. 3 and in a compressed configuration in accordance with embodiments of the present invention;

FIG. 4C illustrates a compression arrangement deployed at a ganglion of the abdominal aorta and in a pre-compressed configuration in accordance with embodiments of the present invention;

FIG. 4D illustrates a compression arrangement deployed at the ganglion shown in FIG. 4C in a compressed configuration in accordance with embodiments of the present invention;

FIGS. 5A-5D illustrate a portion of a renal nerve having a nominal shape, which is shown in FIGS. 5A and 5B, and a compressed shape, which is shown in FIGS. 5C and 5D;

FIG. 6A illustrates a compression arrangement implemented as a fastener in accordance with embodiments of the present invention;

FIG. 6B illustrates a compression arrangement implemented as a rivet in accordance with embodiments of the present invention;

FIG. 6C shows a tissue piercing feature of a compression arrangement implemented in accordance with embodiments of the present invention;

FIG. 6D shows implantation of several compression arrangements distributed in a spaced relationship along a wall of the renal artery, the pattern defined by the distribution of compression arrangements following a generally spiral or helical shape in accordance with embodiments of the present invention;

FIGS. 7A-7C illustrate an apparatus for implanting a compression arrangement into a target vessel wall using an intravascular approach in accordance with embodiments of the present invention;

FIGS. 7D-7F illustrate an apparatus for implanting a compression arrangement into a target vessel wall using an extravascular approach in accordance with embodiments of the present invention;

FIGS. 8A, 8B, and 9 illustrate extravascular cuff implementations that place nerves of the renal artery in compression in accordance with embodiments of the present invention;

FIGS. 10A-10C illustrate an apparatus for positioning a compression cuff on a renal artery using an extravascular approach in accordance with embodiments of the present invention;

FIG. 11 shows a variation of a multiple-cuff compression mechanism according to embodiments of the present invention;

FIGS. 12A and 12B illustrate an embodiment of a compression arrangement configured to compress nerves of a vessel using an intravascular stent and an extravascular filament in accordance with embodiments of the present invention; and

FIG. 13 illustrates different configurations of compression arrangements according to embodiments of the present invention deployed together on a patient's renal artery.

While the invention is amenable to various modifications and alternative forms, specifics thereof have been shown by way of example in the drawings and will be described in detail. It is to be understood, however, that the intention is not to limit the invention to the particular embodiments described. On the contrary, the intention is to cover all modifications, equivalents, and alternatives falling within the scope of the invention as defined by the appended claims.

### DETAILED DESCRIPTION

In the following description, references are made to the accompanying drawings which illustrate various embodiments of the invention. It is to be understood that other embodiments may be utilized, and structural and functional changes may be made to these embodiments without departing from the scope of the present invention.

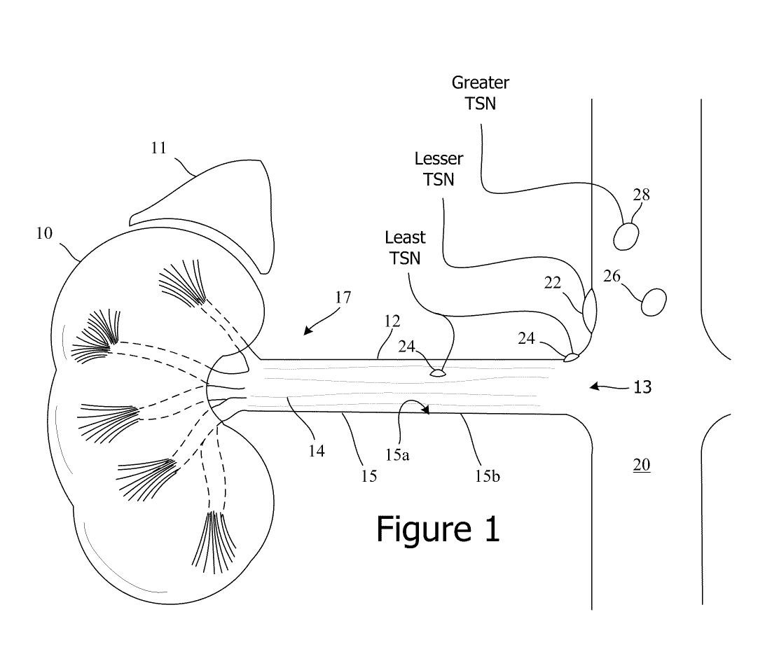

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the present invention. The renal artery 12 is purposefully shown to be disproportionately larger than the right kidney 10 and abdominal aorta 20 in order to facilitate discussion of various features and embodiments of the present disclosure.

The right and left kidneys are supplied with blood from right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

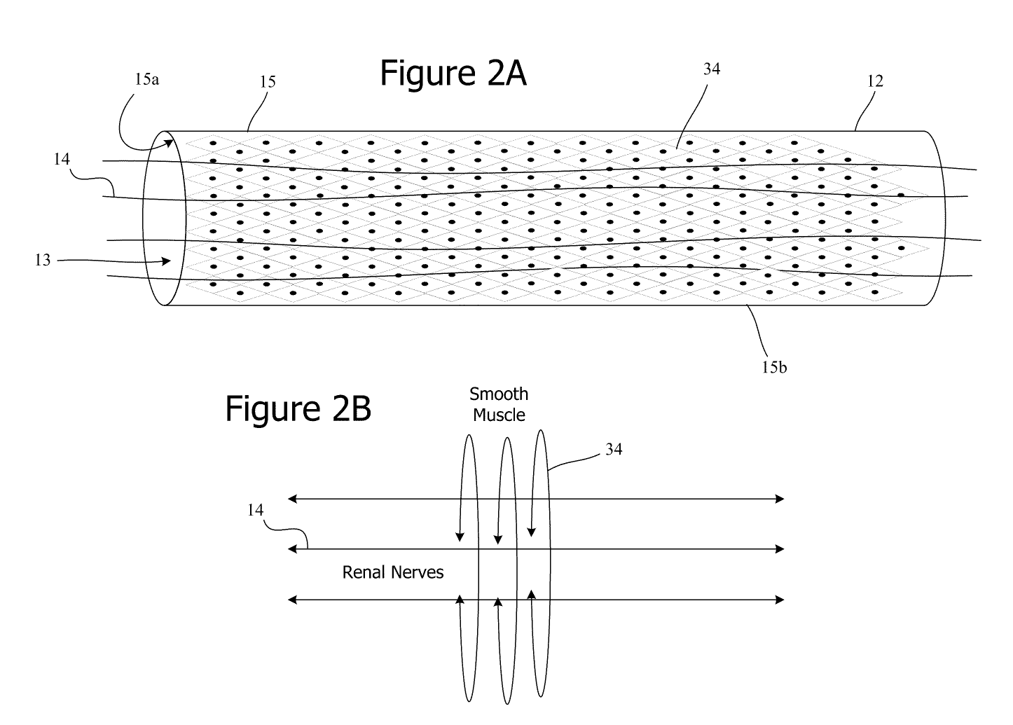

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

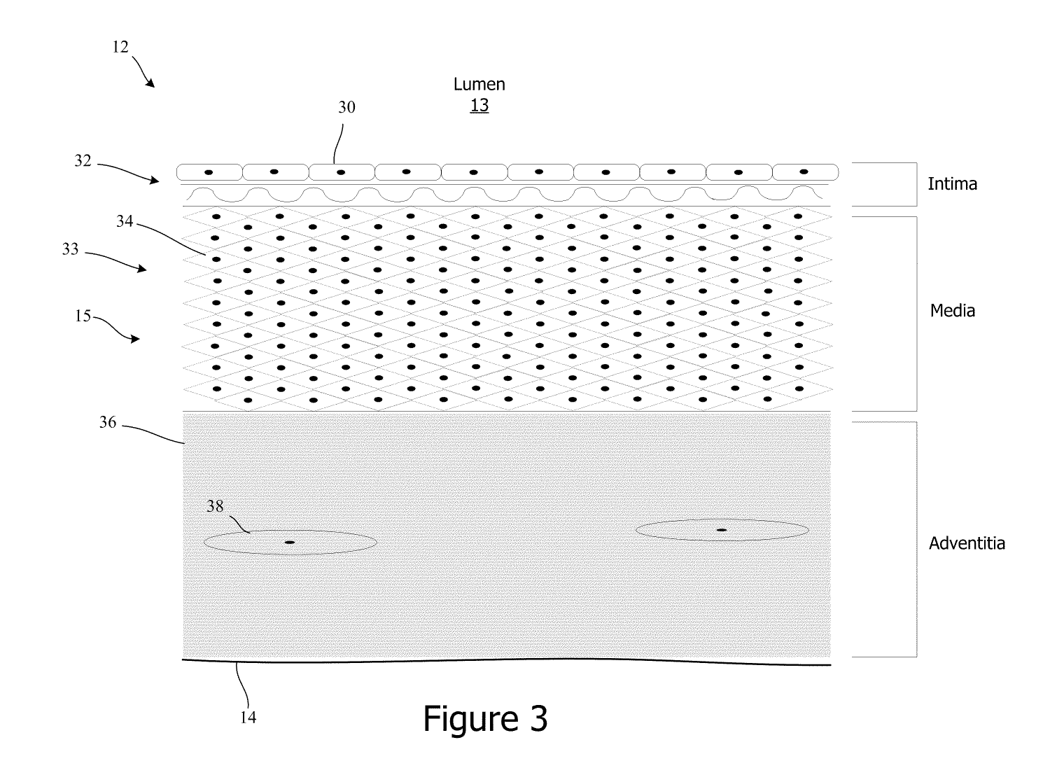

FIG. 3 shows a segment of a longitudinal cross-section through a renal artery 12, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing. A renal nerve 14 is shown proximate the adventitia 36 and extending longitudinally along the renal artery 12. The main trunk of the renal nerves 14 generally lies in or on the adventitia 36 of the renal artery 12, with certain branches coursing into the media 34 to enervate the renal artery smooth muscle 34.

Embodiments of the present invention are directed to arrangements configured to purposefully cause damage to a target nerve or ganglion, such as the renal nerve or aorticorenal or superior mesenteric ganglion, resulting in neuropathic derangement of the function and/or structure of a target nerve or ganglion, preferably by application of compressive force having a defined magnitude. Embodiments of the present invention are directed to mechanical arrangements that are situated relative to a target vessel wall or ganglion and are configured generate a compressive force sufficient to disrupt or, more preferably, terminate renal sympathetic nerve activity while generally preserving the structural integrity of the target vessel wall or ganglion and surrounding tissue. Mechanical arrangements implemented in accordance with the present invention may include an adjustment feature that facilitates control of the magnitude and/or region of application of compressive force imparted to a target nerve or ganglion. Some embodiments of a mechanical arrangement implemented in accordance with the present invention may include an energy, thermal, or drug transfer element or circuit that facilitates transfer of energy (e.g., ultrasonic, RF, microwave), direct thermal (heat or cold) therapy, or a pharmacological agent to the target nerve or ganglion.

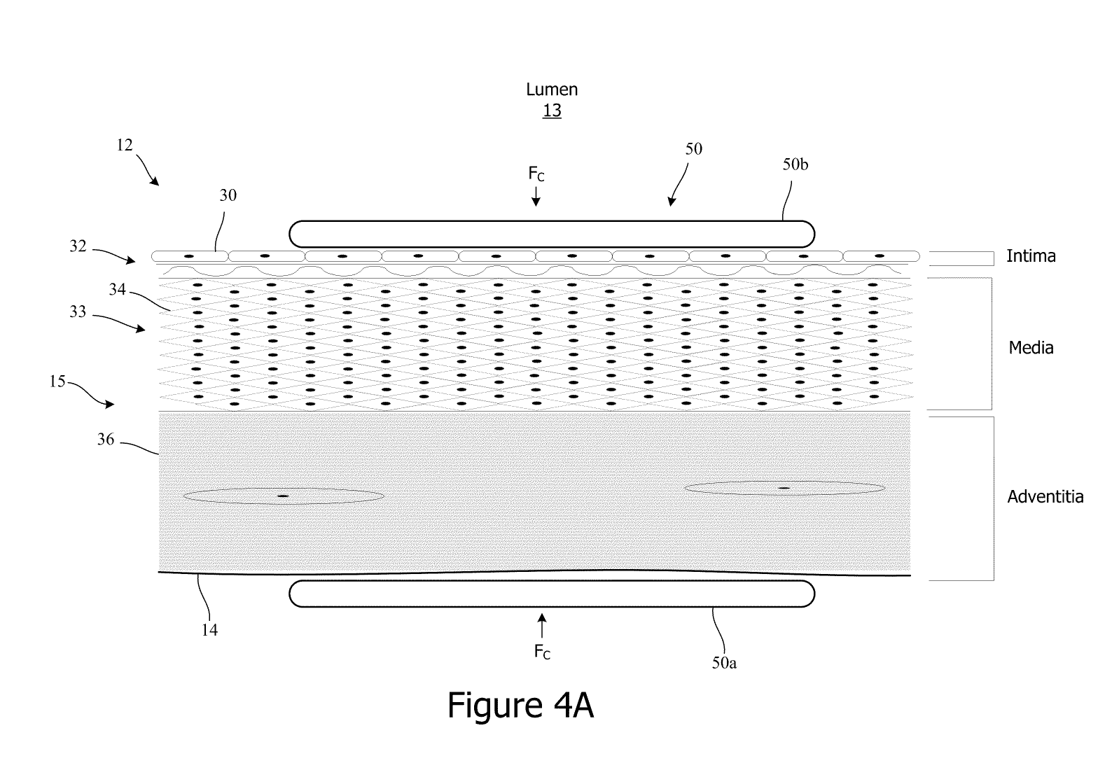

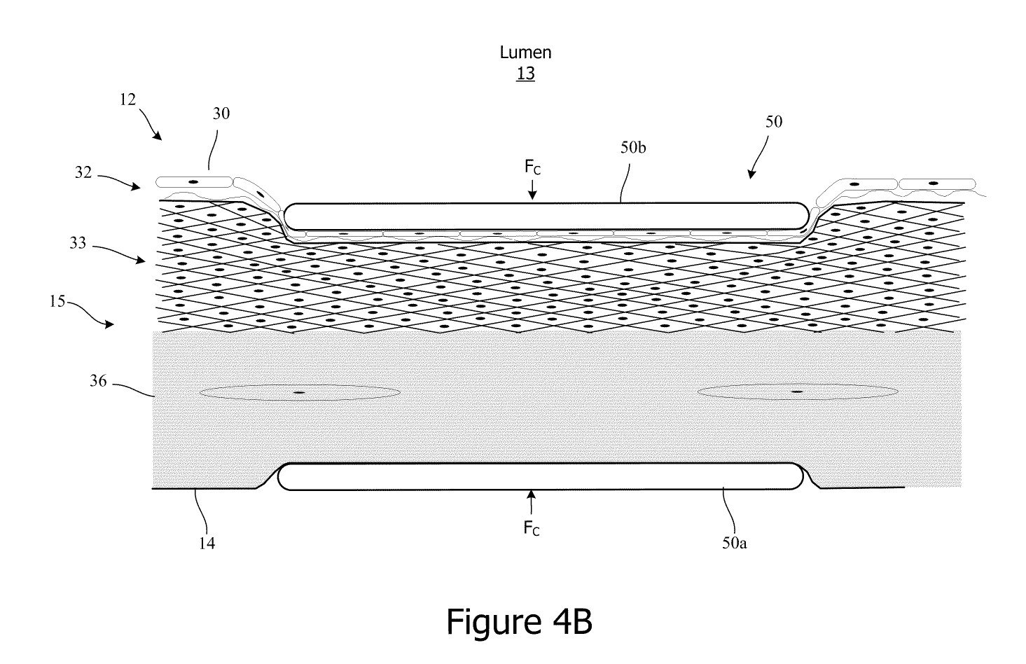

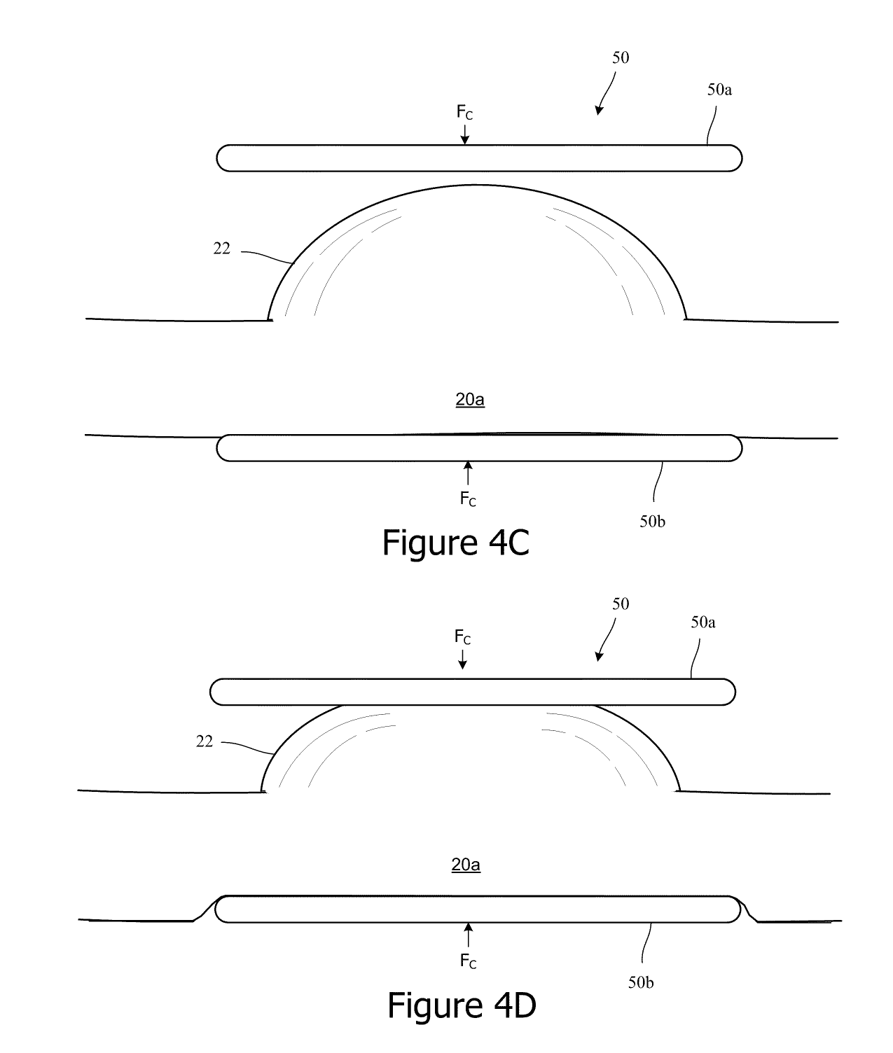

A representative embodiment of an arrangement configured to modify nerve activity along a nerve of a target vessel in accordance with embodiments of the present invention is shown in FIGS. 4A and 4B. The representative embodiment of the compression arrangement 50 shown in FIGS. 4A and 4B is configured to mechanically treat the renal artery 12 in order to disrupt or terminate renal sympathetic nerve activity. Preferably, embodiments of the compression arrangement 50 according to FIGS. 4A and 4B are configured for mechanically treating the renal artery 12 to irreversibly terminate all renal sympathetic nerve activity.

FIG. 4A shows a compression arrangement 50 that includes an extravascular element 50a and an intravascular element 50b. FIG. 4A illustrates the state of the compression arrangement 50 prior to compression of the target vessel. FIG. 4B illustrates the compression arrangement 50 in its deployed state, in which a portion of a wall of the target vessel is forcibly squeezed or pinched by compressive force, F_{C}, generated by the compression arrangement 50. The magnitude of the compressive force, F_{C}, generated by the compression arrangement 50 is preferably calibrated to provide a desired degree of nerve activity cessation while limiting damage to the target vessel wall.

The extravascular and intravascular elements 50a and 50b mechanically cooperate to disrupt nerve conduction along nerve fibers 14 extending along a target vessel, such as the renal artery 12. In some embodiments, the extravascular and intravascular elements 50a and 50b are mechanically coupled to one another. In other embodiments, the extravascular and intravascular elements 50a and 50b are not mechanically coupled to one another, but cooperate to mechanically disrupt nerve conduction along nerve fibers 14.

Mechanically treating nerve fibers of a target vessel, such as a renal artery 12 as shown in FIGS. 4A and 4B, is preferably provided by a compression arrangement 50 that places one or more nerve fibers of a target vessel in compression, such as the renal nerve 14. Placing fibers of a nerve in compression results in an inability of the nerve fiber to transmit nerve impulses. The extent and permanency of nerve impulse transmission interruption along a target nerve, such as the renal nerve, may be tailored to achieve a desired reduction in sympathetic nerve activity (including a partial or complete block) and to achieve a desired degree of permanency (including temporary or irreversible injury). A compression arrangement 50 of the present invention may be implemented to generate a predefined magnitude of compressive force for application over a specified region of nerve tissue sufficient to achieve a desired reduction in nerve activity, such as irreversible loss of renal sympathetic nerve activity.

A representative embodiment of an arrangement configured to modify sympathetic nerve activity at a ganglion, such as the aorticorenal ganglion 22, in accordance with embodiments of the present invention is shown in FIGS. 4C and 4D. It is understood that arrangements configured to modify nerve activity at a ganglion in accordance with the present invention may be configured for deployment at any ganglion, particularly those that influence renal sympathetic nerve activity, and that reference to the aorticorenal ganglion 22 in FIGS. 4C and 4D is for non-limiting illustrative purposes only.

The compression arrangement 50 shown in FIGS. 4C and 4D includes an extravascular element 50a and an intravascular element 50b. In some embodiments, the extravascular and intravascular elements 50a and 50b are mechanically coupled to one another. In other embodiments, the extravascular and intravascular elements 50a and 50b are not mechanically coupled to one another, but cooperate to mechanically disrupt nerve conduction at the target ganglion 22. As illustrated, intravascular element 50b is positioned at an inner wall location of the abdominal aorta 20a, and extravascular element 50b is positioned adjacent the aorticorenal ganglion 22 located on the outer wall of the abdominal aorta 20a.

FIG. 4C illustrates the state of the compression arrangement 50 prior to compression of the target ganglion 22. FIG. 4D illustrates the compression arrangement 50 in its deployed state, in which the target ganglion 22 is forcibly squeezed or pinched by compressive force, F_{C}, generated by the compression arrangement 50. The magnitude of the compressive force, F_{C}, generated by the compression arrangement 50 is preferably calibrated to provide a desired degree of nerve activity cessation at the target ganglion 22 while limiting damage to surrounding tissue. Compression arrangements 50 of the same or different configuration may be used cooperatively for mechanically treating nerve fibers and ganglia that influence renal sympathetic nerve activity, preferably by imparting localized compression of sufficient magnitude to terminate renal sympathetic nerve activity.

In some embodiments, the magnitude of compressive force imparted to one or more nerve fibers and/or ganglion of a target vessel may be modified to control or change the level of sympathetic nerve activity. The compression arrangement 50 may incorporate an adjustment feature that facilitates direct modification of compressive force imparted by the compression arrangement 50, such as by use of a physician tool that couples to a compression adjustment mechanism of the compression arrangement 50. An adjustment feature may be integral to the compression arrangement 50 that facilitates remote modification of compressive force imparted by the compression arrangement 50, such as by use of a powered adjustment mechanism that receives or harvests energy.

In embodiments directed to treating the renal artery 12, one or several mechanical compression arrangements 50 are preferably positioned on the renal artery 12 in accordance with a predetermined pattern that provides for termination of all renal sympathetic nerve activity. The predetermined pattern is preferably defined by positioning or distribution of one or more compression arrangements 50 so that at least one complete turn or revolution of the renal artery 12 is treated by of one or more compression arrangement 50.

Positioning or distribution of one or more compression arrangements 50 according to a predetermined pattern encompassing at least one complete turn or revolution of the renal artery 12 advantageously facilitates a “one-shot” denervation therapy of the renal artery or other vessel in accordance with embodiments of the present invention. The term “one-shot” treatment refers to treating the entirety of a desired portion of a vessel without having to move the compression implement or arrangement to other vessel locations in order to complete the treatment procedure (as is the case for a step-and-repeat denervation therapy approach).

A one-shot treatment approach of the present invention advantageously facilitates delivery of denervation therapy that treats at least one location of each nerve fiber extending along a target vessel, such as the renal artery, without having to reposition the compression arrangement(s) 50 during denervation therapy delivery. Embodiments of the present invention allow a physician to position a compression arrangement 50 at a desired vessel location, and completely treat the vessel without having to move the compression arrangement 50 to a new vessel location. A one-shot treatment approach of the present invention also facilitates delivery of denervation therapy that treats one or more ganglia of a target vessel, such as one or more ganglia of the abdominal aorta, without having to reposition the compression arrangement 50 during denervation therapy delivery. It is to be understood that devices and methods that utilize a compression arrangement 50 of the present invention provide advantages and benefits other than facilitating one-shot treatment of a vessel or ganglion, and that compression treatment arrangement patterning that enables one-shot vessel or ganglion treatment is not a required feature in all embodiments.

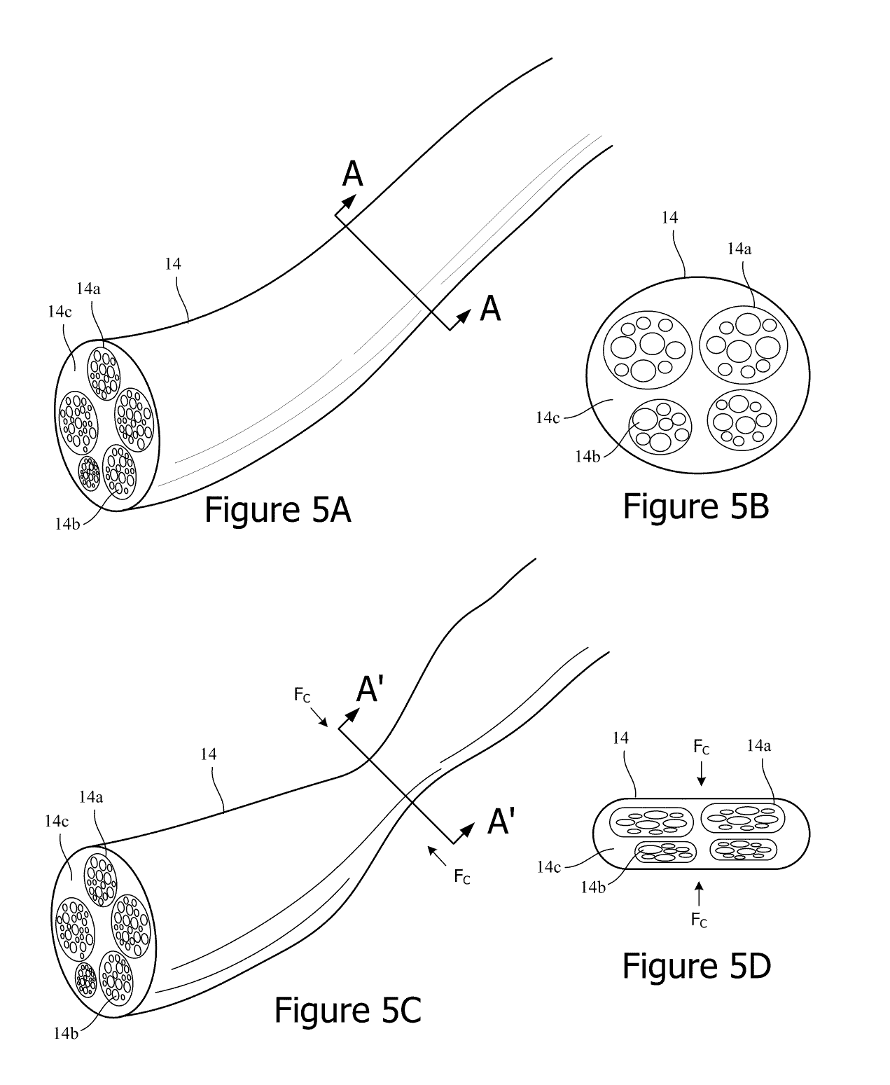

FIGS. 5A-5D illustrate a portion of a renal nerve 14 having a nominal shape, as shown in FIGS. 5A and 5B, and a compressed shape, as shown in FIGS. 5C and 5D. FIG. 5B is a cross-sectional view of FIG. 5A taken along the section A-A, and FIG. 5D is a cross-sectional view of FIG. 5C taken along the section A′-A′. The portion of the renal nerve 14 shown in FIGS. 5A-5D includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus. Nerve fiber regeneration and re-innervation may be permanently compromised by applying a sufficiently large injurious force that physically disrupts or separates the endoneurium tube.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation. Demyelination of axons is associated with various neurological symptoms caused by certain diseases and can result from compressive force injuries to the nerves.

In accordance with various embodiments, one or several compression arrangements 50 of the same or different configuration may be deployed on the renal artery 12 and/or ganglion of the renal artery 12 or abdominal aorta 20 to terminate transmission of action potentials along nerve fibers 14b of the renal artery 12. Compressive force generated by a compression arrangement 50 is imparted to renal nerve fibers 14b and interrupts polarization and/or depolarization cycles associated with normal communication of electric impulses across cell membranes of the nerve fibers 14b during the transmission of nerve impulses along the renal artery 12 and/or across the cell membranes of the smooth muscle of the renal artery 12 and its bed of arterioles during contraction. The degree of interruption of action potential transmission along renal nerve fibers 14b may be varied by delivering an appropriate magnitude of compressive force to the renal nerve fibers 14b via the compression arrangements 50.

In some embodiments, the compression arrangement 50 may be implemented to cause transient and reversible injury to renal nerve fibers 14b. In other embodiments, the compression arrangement 50 may be implemented to cause more severe injury to renal nerve fibers 14b, which may be reversible if compressive force is reduced or removed in a timely manner. In further embodiments, the compression arrangements 50 may be implemented to cause severe and irreversible injury to renal nerve fibers 14b, resulting in permanent cessation of renal sympathetic nerve activity. For example, a compression arrangement 50 may be calibrated or adjusted to produce a clamping or pinching force on a renal nerve fiber 14b sufficient to physically separate the endoneurium tube of the nerve fiber 14b, which can prevent regeneration and re-innervation processes.

By way of example, and in accordance with Seddon's classification as is known in the art, a compression arrangement 50 may be implemented to interrupt conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process that results when a nerve fiber 14b is compressed, crushed or severed, in which the part of the axon separated from the neuron's cell nucleus degenerates. This process is also known as anterograde degeneration. Neurapraxia is the mildest form of nerve injury that may be imparted to renal nerve fibers 14b by one or more compression arrangements 50 of the present invention.

A compression arrangement 50 may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b are preserved. Because axonal continuity is lost, Wallerian degeneration occurs. Axonotmesis is usually the result of a more severe compressive injury, crush or contusion of a nerve fiber 14b than neurapraxia. Recovery from axonotmesis occurs only through regeneration of the axons, a process requiring time on the order of several weeks or months. Electrically, the nerve fiber 14b shows rapid and complete degeneration. If the force creating axonotmesis nerve fiber damage is removed in a timely fashion, the axon may regenerate, leading to recovery. Regeneration and re-innervation may occur as long as the endoneural tubes are intact.

A compression arrangement 50 may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis results from severe contusion, compression, stretching or laceration of a nerve fiber 14b. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

According to the first degree of nerve injury in the Sunderland system (analogous to Seddon's neurapraxia), compression of a nerve, such as the renal nerve 14, results in minimal loss of continuity, local conduction block, and possible focal demyelinization. Recovery of the nerve fiber 14b is usually complete within two to three weeks after removal of compressive force. With second degree nerve injury according to the Sunderland System (analogous to Seddon's axonotmesis), compression of a nerve 14 results in injury to axon and the supporting encapsulating tissue structures 14c (particularly the endoneurium and perineurium). Wallerian degeneration occurs, with axon recovery occurring at about 1 mm per day (typically 0.5-5 mm/day), usually requiring more than 18 months to reach the target tissue.

Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

The amount of compressive force required to achieve a desired reduction of renal sympathetic nerve activity may be determined for a particular patient or from use of human and/or other mammalian studies. For example, a nerve cuff electrode arrangement may be situated on the renal artery of a hypertensive patient that measures nerve impulses transmitted along renal nerve fibers. By way of further example, one or more physiological parameters that are sensitive to changes in renal sympathetic nerve activity may be monitored, and the amount of compressive force required to achieve a desired reduction in renal sympathetic nerve activity may be determined based on measured changes in the physiological parameter(s). Suitable apparatuses for these purposes are disclosed in commonly owned U.S. Patent Publication No. 2008/0234780 and in U.S. Patent Publication No. 2005/0192638, which are incorporated herein by reference.

The nerve cuff electrode arrangement may be integral to the compression arrangement 50 or implemented as a separate structure. Nerve activity measurements may be obtained during placement or implantation of the compression arrangement 50 on the renal artery 12. As discussed previously, it is considered desirable to place/implant the compression arrangement(s) 50 on the renal artery 12 so that at least one complete turn or revolution of the renal artery wall is subject to treatment. With the compression arrangement(s) 50 properly positioned and the renal nerve fibers placed in compression, nerve activity may be monitored using the nerve cuff electrode arrangement to ensure that the compression arrangement(s) 50 compresses the renal nerve fibers sufficiently to attenuate or terminate renal sympathetic nerve activity.

In some embodiments, the compressive force produced by the compression arrangement 50 is alterable during or after placement on the renal artery 12. A desired degree of attenuation in renal nerve activity may be selected by appropriate adjustment of the compression generating mechanism of the compression arrangement 50. In other embodiments, the compressive force produced by the compression arrangement is pre-established to achieve a desired degree of attenuation or termination of renal sympathetic nerve activity. Selecting or controlling the compressive force generated by a compression arrangement 50 advantageously facilitates experimentation and titration of a desired degree and permanency of renal sympathetic nerve activity cessation.

For example, some embodiments of a compression arrangement 50 may be implemented to generate a minimum level of compressive force. This minimum threshold level of renal nerve compression is preferably sufficient to block all renal sympathetic nerve activity and cause a minimum degree of renal nerve damage, consistent with neuropraxia for example. In other embodiments, a compression arrangement 50 may be implemented to generate an intermediate level of compressive force. This intermediate threshold level of renal nerve compression is preferably sufficient to block all renal sympathetic nerve activity and cause an intermediate degree of renal nerve damage, consistent with axonotmesis for example.

In further embodiments, a compression arrangement 50 may be implemented to generate a high level of compressive force. This high threshold level of renal nerve compression is preferably sufficient to block all renal sympathetic nerve activity and cause a high degree of renal nerve damage, consistent with neurotmesis for example. These threshold levels of renal nerve compression may be determined empirically for a patient or by use of human or other mammalian studies. Similar threshold levels of compression may be determined for various ganglia that influence renal sympathetic nerve activity, and compression arrangements 50 may be implemented accordingly for attenuating or terminating nerve activity at various ganglia.

A compression arrangement 50 in accordance with embodiments of the present invention may be implemented to cause localized ischemia of renal nerves. It has been suggested that about 30-60 mmHg of pressure applied to a nerve is sufficient to block axonal blood flow, and that about 60-120 mmHg of pressure applied to a nerve is sufficient to block intraneural blood flow. Chronic application of pressure at appropriate levels leads to perinodal demyelization. Ischemia has been found to occur in a nerve subjected to compressive force in about 15 to 45 minutes, resulting in reversible neuropoxia. When a nerve is subjected to compression for a duration greater than 8 hours, the resulting ischemia has been found to cause irreversible nerve damage (e.g., neurotmesis).

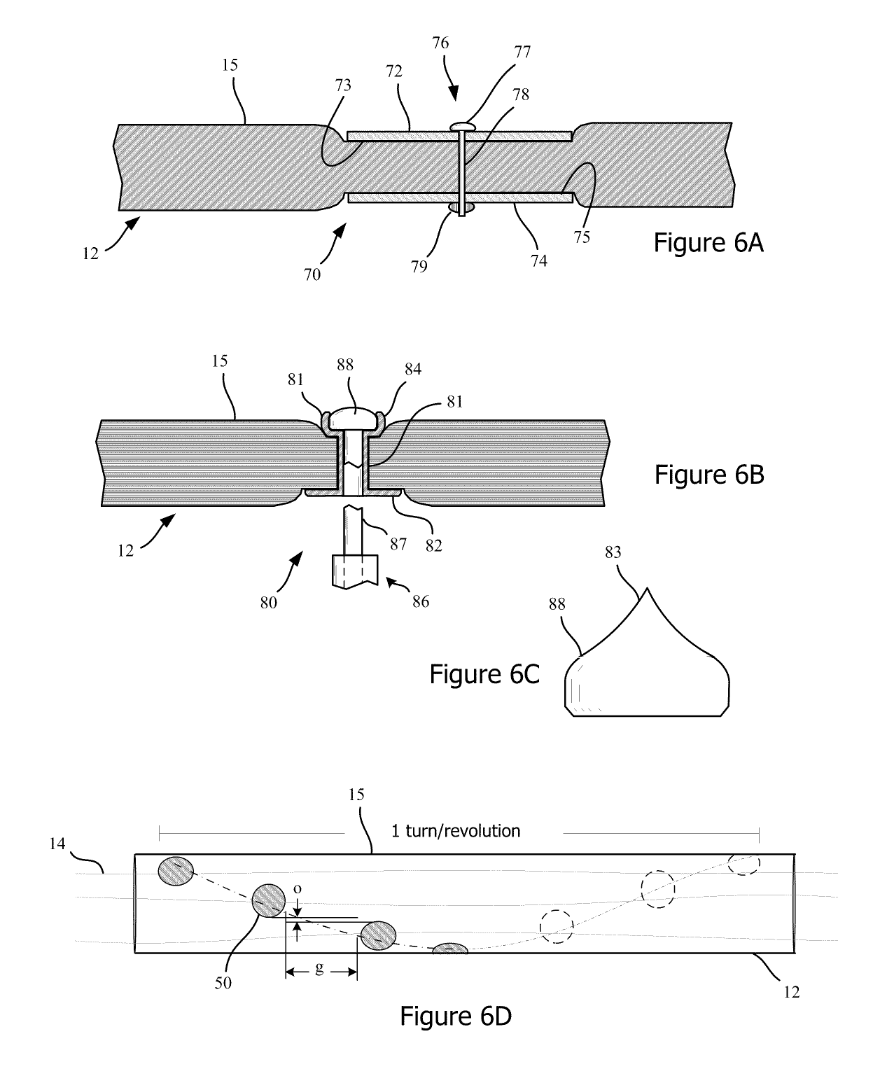

Turning now to FIGS. 6A-6E, various embodiments of a compression arrangement in accordance with the present invention are illustrated. FIG. 6A illustrates a compression arrangement implemented as a fastener 70 in accordance with embodiments of the present invention. The fastener 70 is shown in a deployed state on a wall portion of a target vessel, such as a wall portion 15 of the renal artery 12. The fastener 70 includes a first member 72 having a first contact surface 73 configured to contact a first region of a renal artery 12, which may be an inner wall surface of the renal artery 12. The fastener 70 includes a second member 74 having a second contact surface 75 configured to contact a second region of the renal artery 12, which may be an outer wall surface of the renal artery 12. In some embodiments, the first and second members 72, 74 are formed from a flexible material and have a collapsible configuration that facilitates passage of the fastener 70 through the lumen of a delivery catheter or instrument, and through an access hole created in the wall 15 of the renal artery 12. In other embodiments, the first and second members 72, 74 are formed from relatively rigid or semi-rigid material and incorporate a hinge (e.g., a living hinge) or collapse mechanism that facilitates passage of the fastener 70 through the delivery catheter or instrument lumen and through the renal artery access hole.

The fastener 70 further includes a compression arrangement 76 that mechanically couples the first member 72 and the second member 74, and facilitates maintenance of the first and second contact surfaces 73, 75 in an opposed spaced relationship with respect to one another when in a deployed configuration. The compression arrangement 76 shown in FIG. 6A includes a tension element 78 coupled to first and second heads 77, 79 that mechanically retain the first and second members 72, 74 in a substantially co-planer orientation when in the deployed configuration. The compression arrangement 76 is configured to impart a force to the first and second contact surfaces 73, 75 sufficient to place a wall portion 15 of the renal artery 12 in compression sufficient to achieve a desired reduction or cessation of renal sympathetic nerve activity.

FIG. 6B illustrates a fastener arrangement implemented as a rivet 80. The rivet 80 shown in FIG. 6B is implemented as a blind rivet, such as a blind break-mandrel rivet. The rivet 80 is shown to include a rivet body 81 and a mandrel 86. The rivet body 81 includes a rivet head 82 and an upset head 84, which is configured to capture the mandrel head 88. To implant the rivet 80 in the wall portion 15 of the renal artery 12, the rivet 80 is placed into an implantation implement of a catheter, for intravascular implantation, or a laparoscope or thoracoscope, for extravascular implantation, as will be described in greater detail hereinbelow.

The rivet 80 is advanced through the renal artery wall 15 so that the renal artery wall portion 15 is captured between the rivet head 82 and the upset head 84 formed when the mandrel head 88 is drawn into the distal end of the rivet body 81. Activating the implantation implement pulls the rivet's mandrel 86, drawing the mandrel head 88 into the blind end of the rivet body 81. This action forms the upset head 84 on the rivet body 81 and securely clamps down on the renal artery wall portion 15 with a predetermine level of compression. When the mandrel 86 is pulled and/or twisted with sufficient force, the mandrel 86 reaches its predetermined break-load, with the spent portion 87 of the mandrel 86 breaking away and being withdrawn from the set rivet 80.

In some embodiments, a small hole is created in the wall of the renal artery to provide transvascular access for the rivet 80. In other embodiments, the mandrel head 88 shown in FIG. 6B or one of the first and second heads 77, 79 shown in FIG. 6A may incorporate a tissue piercing tip 83 that is used to create the access hole in the renal artery wall, as is shown in FIG. 6C (e.g., a self-piercing rivet). The tissue piercing tip 83 may be formed of a material that slowly dissolves so as to blunt the sharp tip 83 over time.

The rivet 80 may be implemented as a tri-fold blind rivet. A tri-fold blind rivet advantageously applies the rivet's clamping force over an increased area, reducing the risk of perforating or otherwise damaging the renal artery wall 15. In some embodiments, the fastener 70 or rivet 80 may be configured as, or incorporate features of, a septal defect repair patch, such as those disclosed in U.S. Patent Publication No. 2004/0019348, which is incorporated herein by reference. It is noted that a purse string suture or other tissue-gathering apparatus may be applied to the artery wall 15 surrounding the fastener 70 or rivet 80 and tightened to prevent blood from perfusing through the access hole created in the renal artery wall 15.

The fastener 70 and rivet 80 show in FIGS. 6A-6C may be respectively configured for implantation at a ganglion of the abdominal aorta or renal artery. The tissue contacting surfaces of the fastener 70 and rivet 80 may each have a surface area consistent with surface areas of the renal ganglion or a ganglion or ganglia of the abdominal aorta. For example, the tissue contacting surfaces of the fastener 70 and rivet 80 may each have a surface area consistent with surface areas of the renal ganglion or plexus, the superior mesenteric ganglion, the celiac ganglia or plexus, or the aorticorenal ganglion.

The fastener 70 and rivet 80 shown in FIGS. 6A and 6B are formed from a biocompatible material. Different portions of the fastener 70 and rivet 80 may be made with the same or different material. Suitable materials include polyester, expanded polytetrafluorethylene (EPTFE), shape-memory alloys (e.g., Nitinol), and stainless steel, among others.

FIG. 6D shows implantation of several compression arrangements 50 (e.g., fastener 70 or rivet 80) distributed in a spaced relationship along a wall 15 of the renal artery 12. The pattern defined by the distribution of compression arrangements 50 follows a generally spiral or helical shape. Individual compression arrangements 50 are separated by a longitudinal gap, g. A circumferential overlap, o, may be provided between the end of one compression arrangement 50 and the beginning of another compression arrangement 50 to prevent inclusion or formation of a circumferential gap therebetween. The distribution of compression arrangements 50 as shown in FIG. 6D collectively complete at least one revolution or turn of the renal artery 12, ensuring that at least one location of each renal nerve fiber 14 extending along the renal artery 12 is subject to compressive denervation therapy.

Additionally, the distribution of compression arrangements 50 in FIG. 6D minimizes injury to the vessel wall by distributing the individual sites of injury over the area of the vessel wall. In the distribution of FIG. 6D, the zones of tissue injury around each arrangement 50 may not overlap, allowing for a less aggressive healing response that is localized to the individual sites of injury.

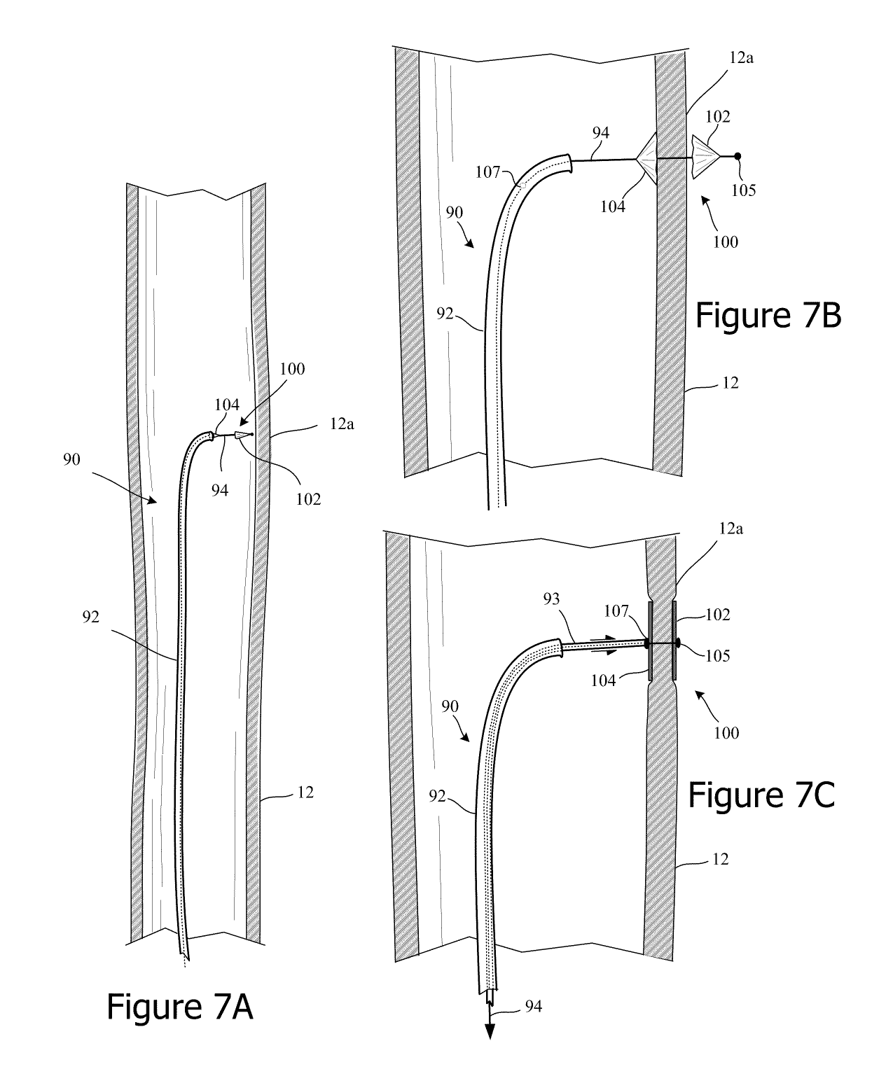

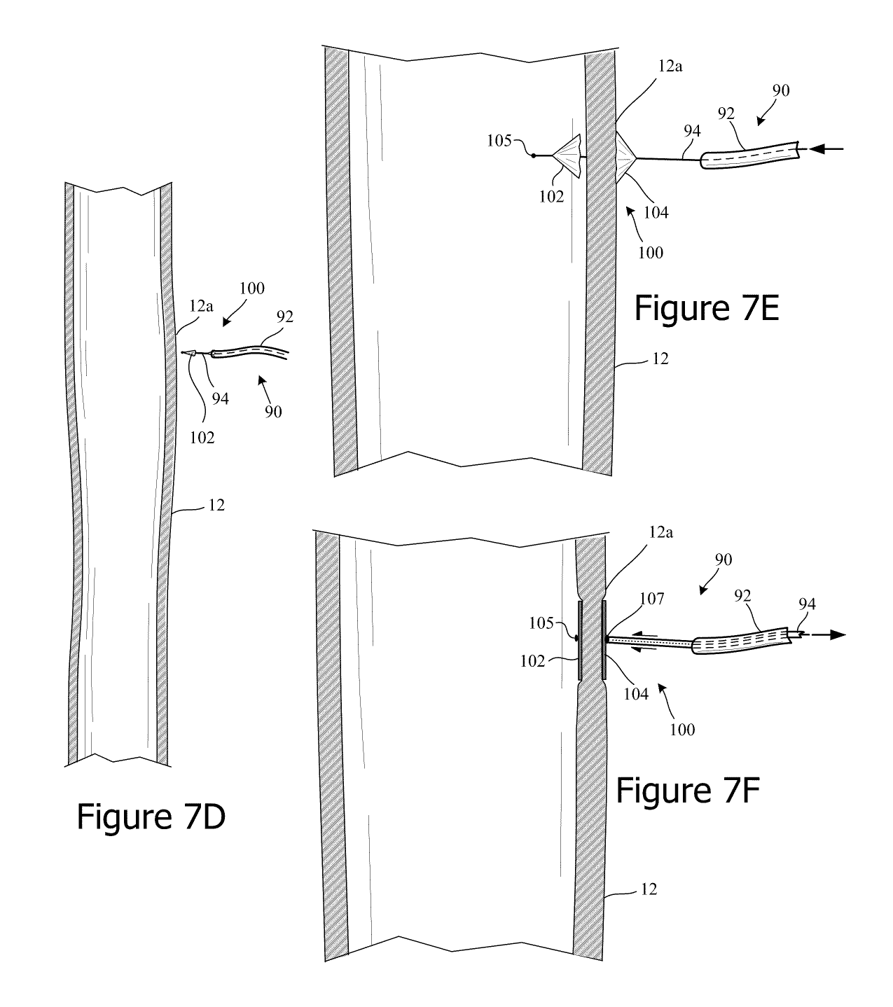

FIGS. 7A-7C illustrate an apparatus for implanting a compression arrangement into a target vessel wall using an intravascular approach in accordance with embodiments of the present invention. The apparatus shown in FIGS. 7A-7C is described in the context of delivering a compression arrangement of the present invention to a target location within the renal artery and implanting the compression arrangement at a wall portion of the renal artery. It is understood that the apparatus shown in FIGS. 7A-7C may be implemented for use in other vessels and structures, including the abdominal aorta, and for implantation at selected ganglia of the abdominal aorta, for example.

FIG. 7A shows a catheter assembly 90 that includes an outer catheter 92 that has been advanced to a renal artery location via an intravascular access path. The outer catheter 92 has a lumen through which a compressive fastener assembly of the present invention is advanced. The outer catheter 92 is shown with a shaped or bent distal end that is orientated about 90 degrees relative to a longitudinal axis of the proximal section of the outer catheter 92. The bend at the distal end of the outer catheter 92 enhances the ease by which a compressive fastener 100 may be implanted in a wall portion 12a of the renal artery 12. The bend at the distal end of the outer catheter 92 may be created after the catheter 92 has been placed in the renal artery 12, such as by removing a stiffening stylet from the catheter lumen, or by engaging push and pull wires contained in the wall of catheter 92.

According to some embodiments, an access hole at the implant site 12a is created using an obturator or wire advanced through the outer catheter 92. The obturator or wire preferably has a sharp end or cutting element that can create an access hole through the renal artery wall 12a. The obturator or wire is withdrawn from the outer catheter 92 after creating the access hole. In other embodiments, a distal member of the compression fastener 100 (e.g., member 105 shown in FIGS. 7B and 7C) may incorporate a tissue penetrating feature, such as tissue piercing tip 83 shown in FIG. 6C. Alternatively, an energy source, for example a radiofrequency or laser source, may be applied at the tip 102 or to distal tip member 105 to assist in puncturing the vessel wall.

The distal tip of the outer catheter 92 may be forced against the inner wall of the renal artery at the implant site 12a using a biasing mechanism (not shown) situated at the distal end of the outer catheter 92, such as a biasing balloon arrangement. Forcing the distal end of the outer catheter 92 against the inner wall of the perforated renal artery may limit or preclude perfusion of blood from the artery through the perforation. A hemostatic sealing member (e.g., sealing o-ring) may be provided at the distal tip (e.g., atraumatic tip) of the outer catheter 92 to enhance sealing at the perforation site.

As is shown in FIG. 7B, the fastener assembly includes a distal member 102, a proximal member 104, and a pull wire 94 which passes through the distal and proximal members 102, 104. The distal and proximal members 102, 104 have a collapsible configuration that allows the fastener assembly to be advanced through the outer catheter 92 and the access hole created in the renal artery wall 12a. The distal and proximal members 102, 104 may have an umbrella-like configuration that collapses in one direction but resists being collapsed in a second direction when deployed.

A distal head 105 is disposed at the distal tip of the pull wire 94. The distal head 105 may be integral to, or fixed at, the distal tip of the pull wire 94. Alternatively, the distal head 105 may have a central bore that allows the distal head 105 to slide along the pull wire 94. In this configuration, the distal tip of the pull wire 94 has an enlarged tip portion that prevents the distal head 105 from sliding past of the distal tip of the pull wire 94. A proximal head 107 is shown recessed within the outer catheter 92 and preferably has a central bore that allows the distal head 105 to slide along the pull wire 94. The proximal head 107 is situated proximal of the proximal member 104 of the fastener assembly.

During the implantation procedure, the fastener assembly is advanced along the lumen of the outer catheter 92 in its collapsed configuration. The distal tip of the pull wire 94, the distal head 105, and the distal member 102 of the fastener 100 are forced through the access hole created in the wall 12a of the renal artery 12, preferably with the distal tip of the outer catheter 92 pressed against the implantation site at the inner wall of the renal artery 12. The proximal member 104 is advanced out of the outer catheter 92 and preferably expands to its deployed state as it exits the distal tip of the outer catheter 92. An inner catheter 93 is advanced over the pull wire 94 and engages the proximal head 107 of the fastener assembly. The proximal head 107 is forced against the proximal member 104, preferably by one pulling on the proximal end of the pull wire 94 with resistance applied to the inner catheter 93.

The proximal head 107 is forced against the proximal member 104 to generate a desired amount of artery wall compression. The proximal head 107 cinches onto the pull wire 94 and the proximal portion of the pull wire 94 is separated from the distal portion, now part of the fastener 100. The proximal portion of the pull wire 94 may be separated from the distal portion by fatiguing the pull wire 94, such as by twisting the pull wire 94 and causing pull wire separation along a pre-scored or weakened region of the pull wire 94. Separation of the pull wire may be achieved by actuation of a mechanical separation means. Alternatively, pull wire separation may occur by applying an electrical current through the pull wire 94 that electrically dissolves a small segment of the wire that is composed of a dissolvable material such as iron. The proximal portion of the pull wire 94, the inner catheter 93, and the outer catheter 92 are withdrawn from the patient, leaving the compressive fastener 100 implanted in the wall 12a of the renal artery 12 (or ganglion of the abdominal aorta).

The amount of compressive force imparted to the renal artery wall portion 12a may be controlled by the amount of tensile force applied to the pull wire 94 during fastener implantation. A sensing arrangement at the proximal end of the pull wire 94 may be used to measure the tensile force applied to the pull wire 94 during fastener implantation. Based on the surface area of the distal and proximal members 102, 104, the tensile force measurements, and other factors, a desired magnitude of artery wall compression may be achieved. It is noted that the cyclical swelling of the renal artery 12 that results from blood pressure pulses may be a factor when selecting the amount of compressible force generated by the fastener 100, to avoid over-pinching the renal artery 12, for example.

It has been found that renal nerve anatomy can be highly variable. In some embodiments, it may be desirable to extend the proximal member 102 a distance beyond the outer wall 12a of the renal artery sufficient to capture perivascular nerves.

For example, the proximal member 102 can be extended between about 10 mm and 20 mm beyond the outer wall 12a of the renal artery. The pull wire 94 can then be retracted proximally so that the proximal member 102 captures perivascular nerves as it is pulled into compressing engagement with the outer wall 12a of the renal artery. This approach provides for the mechanical capture and pinching of any perivascular renal nerves residing beyond the adventitia.

It is understood that this approach and others disclosed herein can be applied at the ostium where renal and aortal arteries meet, and at the TSN region of the aorta, for example.

As was discussed previously, a desired degree and permanency of renal nerve damage may be achieved by selection of the magnitude of compressive force imparted to renal nerve fibers by the fastener 100. For example, a minimum threshold level of renal nerve compression may be selected to achieve cessation of all renal sympathetic nerve activity and cause a minimum degree of renal nerve damage, consistent with neruapraxia. An intermediate threshold level of renal nerve compression may be selected to achieve cessation of all renal sympathetic nerve activity and cause an intermediate degree of renal nerve damage, consistent with axonotmesis. A high threshold level of renal nerve compression may be selected to block all renal sympathetic nerve activity and cause a high degree of renal nerve damage, consistent with neurotmesis.

FIGS. 7D-7F illustrate an apparatus for implanting a compression arrangement into a target vessel wall using an extravascular approach in accordance with embodiments of the present invention. The general description of implanting a compressive fastener 100 using an intravascular technique is largely applicable to implementing an extravascular fastener implantation approach. As such, details of the extravascular approach that are largely equivalent to those of the previously described intravascular approach are omitted for purposes of brevity.

According to an extravascular approach, a percutaneous intrathoracic access procedure, such as a laparoscopic, thoracoscopic, or other minimally invasive surgical procedure, is preferably used to access the outer wall of the renal artery 12. The outer catheter 92 may be more ridged than that of intravascular embodiments to increase kink resistance of the outer catheter 92. Increased kink resistance may be desired since biasing mechanisms, such as a biasing balloon that utilizes back pressure from vessel walls, may have limited usefulness in an extravascular approach. A braid or other structure that enhances kink resistance may be incorporated in the outer catheter 92 shown in FIGS. 7D-7F.

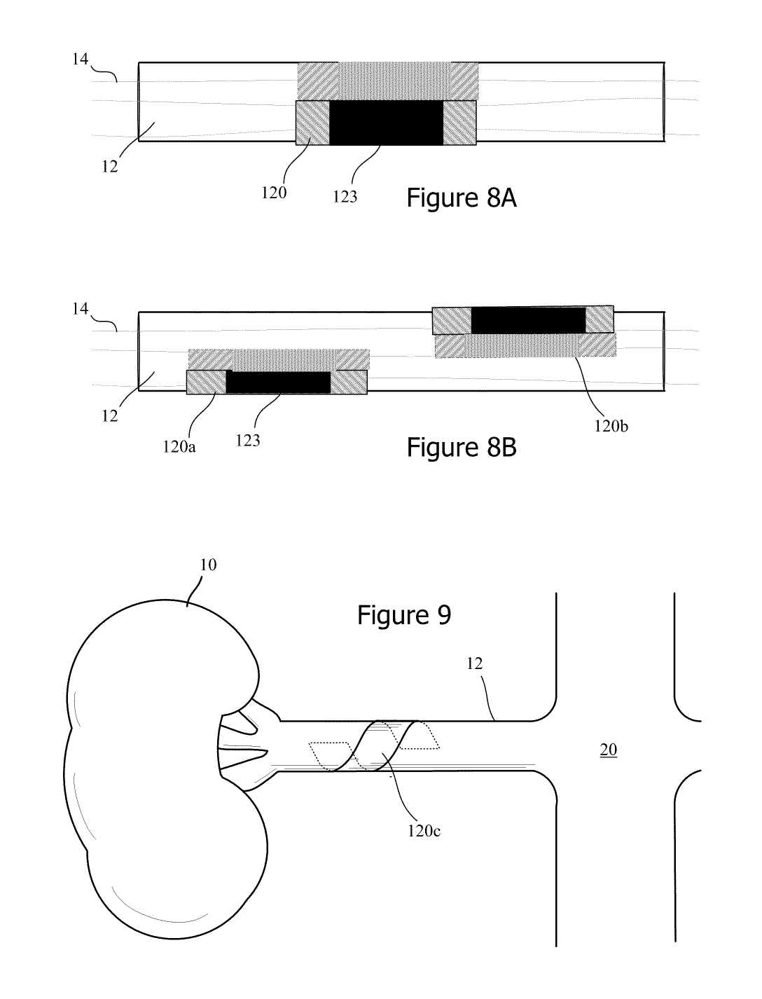

FIGS. 8A, 8B, and 9 illustrate extravascular cuff implementations that place nerves of the renal artery 12 in compression in accordance with embodiments of the present invention. In FIG. 8A, a single cuff 120 is configured for secured positioning on the renal artery 12 and to compress nerves of the renal artery 12 sufficient to reduce or terminate renal sympathetic nerve activity. The cuff 120 is configured to fully envelop the renal artery 12, thereby placing all renal nerve fibers 14 extending along the renal artery 12 in compression.

In FIG. 8B, two cuffs 120a, 120b are configured for secured positioning on the renal artery 12. Cuffs 120a and 120b typically cover artery 12 overall circumferentially to ensure that all nerves 14 of the renal artery 12 are subject to compression sufficient to reduce or terminate renal sympathetic nerve activity. It can be seen in FIG. 8B that the two compression cuffs 120a and 120b together cover the circumference of the renal artery 12 (2 cuffs encompassing at least 180° each for at least 360° of coverage). The two cuffs 120a and 120b are preferably fashioned to cover more than 180° of the renal artery's circumference.

In this configuration, the opposing ends of each cuff 120a and 120b can be pulled away from one another to expand the cuffs 120a and 120b when being positioned around respective portions of the renal artery 12. The cuffs 120a and 120b may then be allowed to clamp down on the renal artery wall with a predefined compressive force, which also serves to maintain secured positioning of the cuffs 120a and 120b on the renal artery wall. The two (or more) cuffs 120a and 120b can by positioned relative to one another on the renal artery 12 to ensure that the cuffs 120a and 120b together place the circumference of the renal artery 12 in compression.

In FIG. 9, a helical or spiral cuff 120c is configured for secured positioning on the renal artery 12 and to compress nerves of the renal artery 12 sufficient to reduce or terminate renal sympathetic nerve activity. In this embodiment, the spiral cuff 120c is formed from a shape-memory material, such as Nitinol, that compresses the renal artery 12 with a predefined force when positioned on the renal artery wall. The helical shape of the spiral cuff 120c serves to place at least one revolution of the renal artery wall in compression.

The cuffs 120-120c preferably incorporate a support element 123, such as a shape-memory element (e.g., a Nitinol element). The support element 123 may be encapsulated in a biocompatible material, such as polyester, EPTFE or silicone. Alternatively, the cuffs 120-120c may be made entirely of a shape-memory alloy. All or part of the tissue contacting surface of the cuffs 120, 120a, 120b, and 120c may incorporate a micromachined pattern or other treatment (e.g., chemical) to form a high friction surface feature that enhances the gripping strength of the cuff 120-120c. Compression cuff embodiments in accordance with the present invention may be implemented to include features of various known vascular and nerve cuff structures, such as those disclosed in U.S. Pat. Nos. 7,584,004; 6,106,477; 5,251,634; and 4,649,936; and in U.S. Patent Publication No. 2008/0004673, which are incorporated herein by reference.

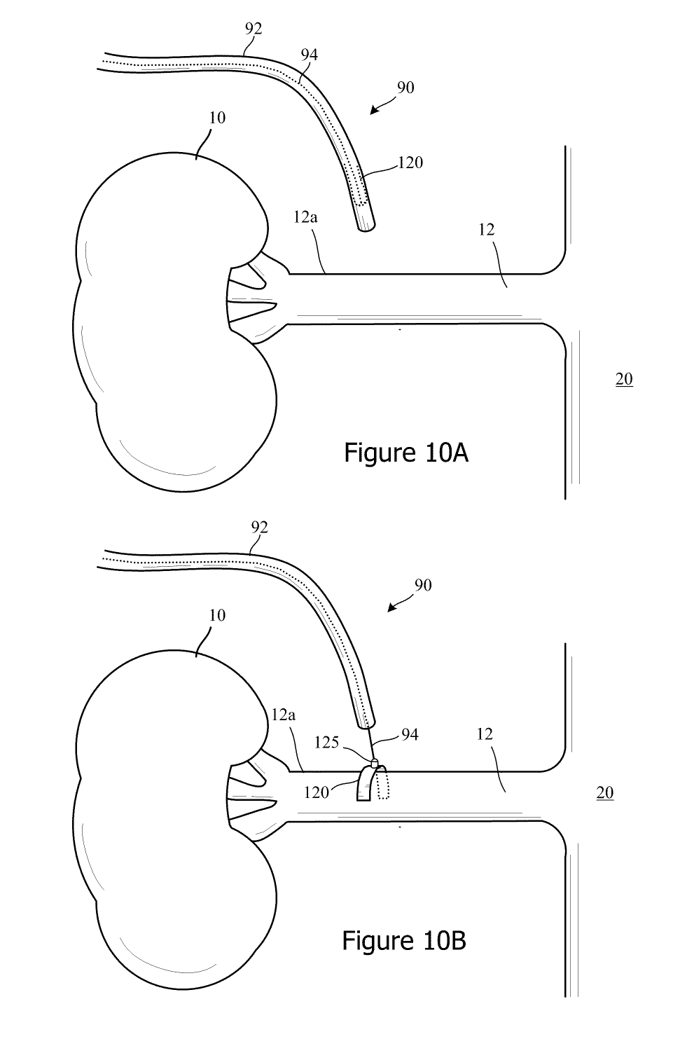

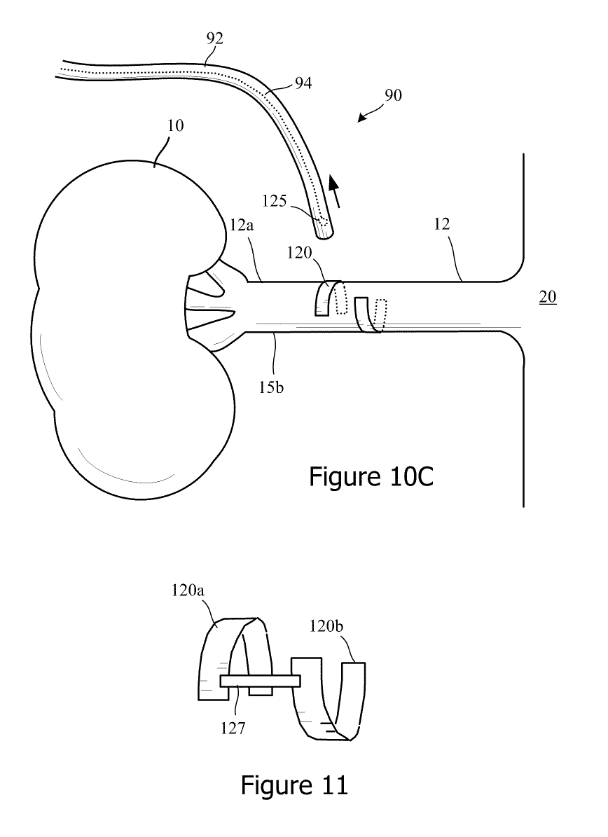

FIGS. 10A-10C illustrate an apparatus 90 for positioning a compression cuff on a renal artery in accordance with an extravascular approach of the present invention. FIG. 10A shows a catheter 92 having an open lumen. The catheter 92 may be a component of a laparoscope, thoracoscope, or other minimally invasive surgical instrument used to access the outer wall 12a of the renal artery 12. A compressive cuff 120 is shown in a compressed non-deployed configuration within the lumen of the catheter 92. The arms of the cuff 120 may be compressed in a backward or forward direction relative to the distal open end of the catheter 92. The compressive cuff 120 is coupled to the distal end of an obturator or wire 94 via a coupler 125. In this non-deployed confirmation, the compressive cuff 120 can be displaced longitudinally through the lumen of the catheter 92 in response to longitudinal displacement of the obturator or wire 94.

FIG. 10B shows the compressive cuff 120 of 10A in a deployed configuration. In FIG. 10B, the compressive cuff 120 has been advanced beyond the distal tip of the catheter 92. As the compressive cuff 120 exits the catheter's distal tip, the compressive cuff 120 assumes it's pre-shaped configuration. The compressive cuff 120 is positioned on an outer wall portion 12a of the renal artery 12. The obturator or wire 94 is disconnected from the compressive cuff 120 by decoupling of the compressive cuff 120 from the obturator or wire 94 at the coupler 125. Various known mechanisms may be employed at the coupler 125 to facilitate engagement and disengagement between the compressive cuff 120 and the obturator or wire 94 after deployment of the compressive cuff 120 on the renal artery wall 12a.

FIG. 10C shows deployment of two compressive cuffs 120 positioned on an outer wall portion 12a of the renal artery 12. The obturator or wire 94 and coupler 125 are shown recessed within the lumen of the catheter 92, and are withdrawn from the patient after placing the compressive cuffs 120 on the renal artery wall. The catheter 92 is also removed from the patient, and the percutaneous access incisions are properly sutured or stapled. FIG. 11 shows a variation of a multiple-cuff compression mechanism according to embodiments of the present invention. The implementation shown in FIG. 11 includes two compressive cuffs 120a and 120b spaced apart from one another connected by a stabilizer member 127. The stabilizer member 127 may be a separate component that is welded or otherwise attached to the two compressive cuffs 120a and 120b, or may be an integral feature of a unitary two-cuff compression mechanism.

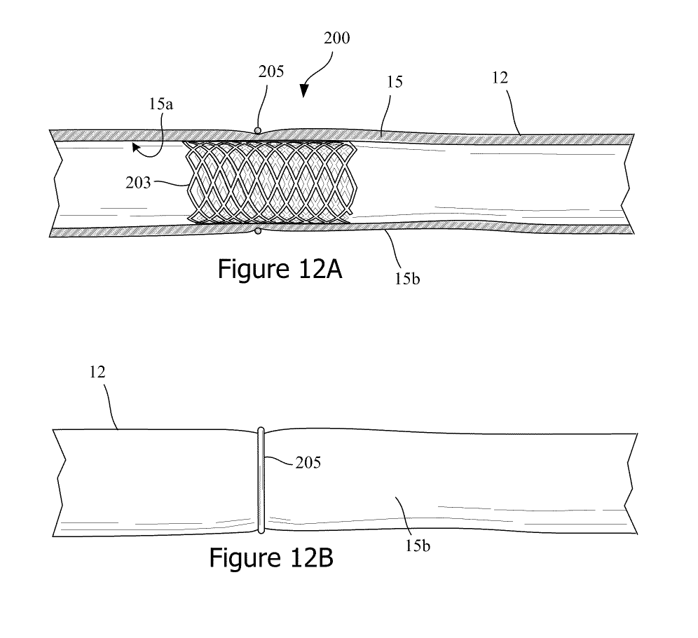

FIGS. 12A and 12B illustrate another embodiment of a compression arrangement configured to compress nerves of a vessel, such as the renal artery, and modify or terminate renal sympathetic nerve activity. The embodiment shown in FIGS. 12A and 12B includes a compression arrangement 200 having an extravascular element and an intravascular element that cooperate to place a portion of a vessel wall in compression. In particular, the compression arrangement 200 shown in FIGS. 12A and 12B includes a extravascular element that is not physically coupled to an intravascular element, yet these elements are configured to cooperatively place a target vessel wall, such as a renal artery wall, in compression at a predefined or adjustable magnitude of compressive force.

The compression arrangement 200 includes a stent 203 dimensioned for deployment in the renal artery 12. Various known intravascular stent delivery apparatuses and techniques may be used to position the stent 203 within the renal artery 12, including those disclosed herein. The stent 203 preferably has a size that allows the outer surface of the stent 203 to engage the inner wall 15a of the renal artery 12. In some configurations, the stent 203 expands when deployed in the renal artery 12 and exerts a radially outward directed force on the wall 15 of the renal artery 12. In other embodiments, the stent 203 need only expand to negligibly engage the wall 15 of the renal artery 12, mostly for positionally stabilizing the stent 203 within the renal artery 12 against dislodgement.

A filament 205 or other extravascular banding element is shown wrapped around the outer wall 15b of the renal artery 12. Various known extravascular delivery apparatuses and techniques may be used to deliver the filament 205 to the renal artery 12 and position the filament 205 relative to the stent 203 residing within the renal artery 12, including those delivery apparatuses and techniques disclosed herein. The filament 205 generates a radially inward directed force when tightened or clamping down on the outer wall 15a of the renal artery 12, which is opposed by the stent 203 positioned immediately adjacent the inner wall 15a of the renal artery 12. In this configuration, the filament 205 and the stent 203 cooperate to place a circumferential wall portion of the renal artery 12 in compression, preferably at a magnitude sufficient to attenuate or terminate all renal sympathetic nerve activity.

In some embodiments, the filament 205 may incorporate a shape-memory element. For example, the filament 205 may be formed from Nitinol. A locking feature may be incorporated at the opposing ends of the filament 205 so that the filament 205 remains securely positioned in the outer wall 15a of the renal artery 12 when deployed. For example, the opposing ends of the filament 205 may be curved or shaped (e.g., U-shaped ends) to capture one another.

In other embodiments, the filament 205 may be a strand of suture or other biocompatible material that is substantially inelastic. The suture or other filament material is preferably selected to provide long-term structural integrity of the filament 205. The suture or other strand of material may be tightened around the outer wall 15a of the renal artery 12 by a physician to a desired tightness.

In further embodiments, the filament 205 may be a strand of suture or other biocompatible material that has elastic properties (e.g., like a rubber-band). In such embodiments, the elastic filament 205 is implemented to generate a desired amount of compression when fitted around the renal artery wall 15 with back pressure provided by the stent 203. A locking arrangement may be disposed on the opposing ends of the elastic filament 205 to ensure positional stability of the filament 205 on the renal artery wall 15.

In some embodiments, the filament 205 may be applied to the external wall of the renal artery from a micro-suture system placed percutaneously within the renal artery 12. In this case, the filament 205 in FIG. 12B may re-enter the artery lumen multiple times in a stitch pattern. One or more rows of stitches may be applied from within the artery to place most or all of the nerves in the artery wall in compression between the filament 205 and the struts of stent 203. The suture line is pulled tight to apply a desired compression force to the renal nerves.

In other embodiments, the filament 205 may consist of a shape memory material, such as Nitinol, that shortens when heated. If the filament 205 comprises a closed loop of electrically conductive shape memory material, such as Nitinol, heat may be generated in the filament 205 by induction of alternating current in the loop from an alternating magnetic field that is applied from outside the patient after the stent 203 and loop 205 have been placed. The shape memory filament 205 may be coated with a thermally insulating material to avoid heating of adjacent tissues when the shape memory filament is heated from an external source.

According to another embodiment, a magnetic compression arrangement may be used to place the renal artery wall in compression. In one configuration, one or more pairs of magnetic compression elements may be placed at intravascular and extravascular locations along the wall of the renal artery 12. The intravascular and extravascular magnet pairs are positioned so that the north and south poles of the extravascular magnet align with the south and north poles of the intravascular magnet. In this orientation, the magnetic fields of the intravascular and extravascular magnets cancel to first order. The magnitude of compressive force generated by a magnet pair is determined by the separation between the magnetic elements, the magnet area, and the magnet material. It is noted that a magnetic compression arrangement of the present invention provides for enhanced safety for patients undergoing MRI evaluation.

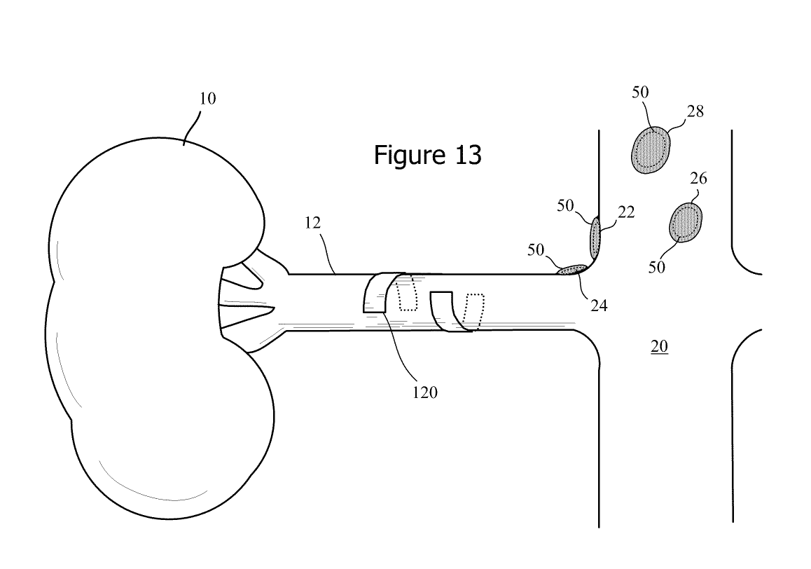

FIG. 13 illustrates different embodiments of compression arrangements of the present invention deployed together on a patient's renal artery 12 and abdominal aorta 20 for attenuating and, preferably, terminating all renal sympathetic nerve activity. In this illustrative embodiment, a compression arrangement 50 (e.g., fasteners, rivets) is implanted at specified locations on the abdominal aorta 20 to cause predefined compressive injury to the superior mesenteric ganglion 26, aorticorenal ganglia 22, celiac ganglia 28, and renal ganglia 24, respectively. A pair of compressive cuffs 120 is shown mounted to the external wall of the renal artery 12 with sufficient coverage to impart a predetermined injurious compressive force to all sympathetic nerves extending along the renal artery 12. Combined use of both renal artery and abdominal aortic ganglia compressive arrangements enhances the efficacy of achieving a desired reduction or termination of renal sympathetic nerve activity.

According to various embodiments, it may be desirable to construct all or portions of a compression arrangement of a type disclosed herein from a biodegradable material or materials. For example, a mechanical crimping apparatus or other compression mechanism can be constructed from biodegradable material that dissolves over a specified duration of time.

In various embodiments, renal nerves and ganglia would likely be irreversibly damaged after being crimped for days or weeks. For a particular patient, a physician may prefer that the crimping/compression mechanism dissolve to prevent long term complications and/or facilitate re-innervation of the renal artery or other target tissue.

Suitable biodegradable crimping or compression arrangements include those with structures constructed iron or magnesium, alloys of iron or magnesium, and/or biodegradable polymers. Suitable biodegradable polymers include biodegradable polyester, polycarbonate, polyorthoester, polyanhydride, poly-amino-acid and/or polyphosphazine, and polylactide with or without an amount of polyisobutylene sufficient to allow the copolymer to be flexed or expanded without cracking. Portions of a biodegradable crimping or compression arrangement according to some embodiments may be formed from biodegradable or bioerodible materials having different composition and/or different erosion rates. Details of various biodegradable materials and structural features that can be useful in constructing biodegradable crimping or compression arrangements according to various embodiments are disclosed in commonly owned U.S. Published Application Nos. 2010/0292776 and 2010/0166820, which are incorporated herein by reference.

The foregoing description of the various embodiments of the invention has been presented for the purposes of illustration and description. It is not intended to be exhaustive or to limit the invention to the precise form disclosed. Many modifications and variations are possible in light of the above teaching. For example, the devices and techniques disclosed herein may be employed in vasculature of the body other than renal vasculature, such as coronary and peripheral vessels and structures. It is intended that the scope of the invention be limited not by this detailed description, but rather by the claims appended hereto.

What is claimed is:

1. A device for mechanically modifying renal sympathetic nerve activity, comprising:
a contact arrangement having a shape that generally conforms to a portion of a renal artery wall and configured for placement at the renal artery wall portion; and
a compression arrangement configured to cooperate with the contact arrangement to place the wall portion of the renal artery in compression sufficient to terminate renal sympathetic nerve activity along the renal artery wall portion.

2. The device of claim 1, wherein one of the contact and compression arrangements is configured for positioning within the renal artery and the other of contact and compression arrangements is configured for positioning on an outer surface of the renal artery.

3. The device of claim 1, wherein each of the contact and compression arrangements is configured for positioning on an outer surface of the renal artery.

4. The device of claim 1, wherein the compression arrangement comprises an actuatable member configured to adjust the force imparted to the contact arrangement.

5. The device of claim 1, comprising one or more sensors configured to sense one or more physiologic parameters that are modified by changes in renal sympathetic nerve activity.

6. The device of claim 1, wherein:
the contact arrangement comprises a cuff member dimensioned to be disposed over an exterior wall portion of a renal artery, the cuff member comprising a contact surface configured to engage the exterior wall portion of the renal artery; and
the compression arrangement comprises a compression element coupled or integral to the cuff member, the compression element and cuff member cooperating to place the wall portion of the renal artery in compression sufficient to terminate renal sympathetic nerve activity along the exterior wall portion of the renal artery.

7. The device of claim 6, wherein the cuff member has a substantially spiral shape.

8. A system for mechanically modifying renal sympathetic nerve activity according to claim 6, comprising:
a biasing device configured for endoluminal deployment within the renal artery and at a location proximate the cuff device; and
the biasing device and the cuff device cooperating to place the wall portion of the renal artery in compression sufficient to terminate renal sympathetic nerve activity along the exterior wall portion of the renal artery.

9. The system according to claim 8, wherein the biasing device comprises a balloon or a stent.

10. A fastener for mechanically modifying renal sympathetic nerve activity, comprising:
a contact arrangement comprising a first element configured to contact an outer wall of a target vessel and a second element configured to contact an inner wall of the target vessel, at least one of the first and second elements having a collapsible configuration that facilitates passage through an access hole developed in the target vessel wall when in the collapsed configuration; and
a force generating arrangement coupled to the contact arrangement and configured to mechanically cooperate with the at least one of the first and second elements to place a wall portion of the target vessel comprising the outer and inner wall in compression sufficient to terminate renal sympathetic nerve activity along the wall portion of the target vessel;
wherein the target vessel comprises at least one of the renal artery and the abdominal aorta.

11. The fastener of claim 10, wherein the fastener comprises a rivet.

12. The fastener of claim 10, wherein the at least one of the first and second elements comprises a collapsible umbrella arrangement that facilitates collapsing and expanding of the at least one of the first and second elements.

13. A delivery system according to claim 10, the delivery system comprising:
a sheath comprising a lumen having a diameter smaller than a cross-sectional diameter of the fastener when the fastener is in a deployed configuration;
the sheath adapted to receive the fastener and compress the first and second elements to at least the lumen diameter; and
a displacement member dimensioned for placement within the lumen and configured to longitudinally displace the fastener within the lumen.

14. A delivery system according to claim 13, comprising an imaging sensor disposed at or near a distal end of the sheath for facilitating implantation of the fastener on the wall of the target vessel.

15. An apparatus for mechanically modifying renal sympathetic nerve activity, comprising:
a stent configured for endoluminal deployment within the renal artery; and
a filament configured for placement around an exterior wall portion of the renal artery and at a location proximate the stent, wherein cooperation between the stent and contraction or shortening of the filament places the wall portion of the renal artery in compression sufficient to terminate renal sympathetic nerve activity along the exterior wall portion of the renal artery.

16. The apparatus of claim 15, wherein the filament is substantially inelastic.

17. The apparatus of claim 15, wherein the filament comprises an elastic material.

18. A device for mechanically modifying renal sympathetic nerve activity, comprising:
a contact arrangement having a shape that generally conforms to a portion of a renal artery wall and configured for placement at the renal artery wall portion;
a compression arrangement configured to cooperate with the contact arrangement to place the wall portion of the renal artery in compression sufficient to terminate renal sympathetic nerve activity along the wall portion of the renal artery; and
a treatment arrangement coupled to the contact arrangement, the treatment arrangement configured to deliver a treatment agent to the renal artery wall portion to facilitate termination of renal sympathetic nerve activity along the renal artery wall portion.

19. The device of claim 18, wherein the treatment arrangement comprises an electrode arrangement configured to receive energy from a source remote from the renal artery wall portion and generate heat that is communicated to the renal artery wall portion.

20. The device of claim 18, wherein the treatment arrangement comprises a mechanism for delivering a pharmacological agent to the renal artery wall portion.

21. The device of claim 1, wherein all or at least a portion of the contact arrangement and the compression arrangement is constructed from one or more biodegradable materials.

22. The device of claim 15, wherein the stent and the filament are constructed from one or more biodegradable materials.
